(19) United States
(12) Patent Application Publication
(10) Pub. No.: US 2025/0333799 A1
(43) Pub. Date: Oct. 30, 2025

(54) GENE SIGNATURE FOR EARLY AGING MESENCHYMAL STEM CELLS AT EARLY PASSAGE

(71) Applicant: The Administrators of the Tulane Educational Fund, New Orleans, LA (US)

(72) Inventors: Kim O'CONNOR, New Orleans, LA (US); Yaozhong LIU, New Orleans, LA (US); Margaret GILER, New Orleans, LA (US); Amanda FOOTE, New Orleans, LA (US)

(21) Appl. No.: 19/192,771

(22) Filed: Apr. 29, 2025

Related U.S. Application Data
(60) Provisional application No. 63/640,198, filed on Apr. 29, 2024.

(51) Int. Cl. C12Q 1/6888; A61K 35/28

(52) U.S. Cl. CPC C12Q 1/6888; A61K 35/28; C12Q 2600/158

(57) ABSTRACT

A method of preparing a therapeutic mesenchymal stem cell composition is described where the MSCs are selected and isolated with a gene signature. The gene signature can be used to provide MSCs that have improved therapeutic properties by removing aging MSCs at early passages.

### PRIOR RELATED APPLICATIONS

This application claims priority to U.S. provisional application 63/640,198, filed Apr. 29, 2024, which is incorporated herein in its entirety for all purposes.

### FEDERALLY SPONSORED RESEARCH STATEMENT

This invention was made with government support under Grant No: CBET-1604129 awarded by the National Science Foundation. The government has certain rights in the invention.

### FIELD OF THE DISCLOSURE

The disclosure generally relates to gene signatures for aging mesenchymal stem cells in early passages.

### BACKGROUND OF THE DISCLOSURE

Mesenchymal stem cells (MSCs) are a powerful tool for wide variety of medical applications. However, MSC aging or even senescence poses challenges to effective MSC therapy, as the cultured cells going through multiple passages would eventually enter a state of permanent cell cycle arrest and stop dividing and undergo functional decline. The senescence can be triggered by stress or aging, characterized by enlarged morphology, decreased proliferation and altered differentiation capacity.

Molecular profiles of mesenchymal stem cells (MSCs) are needed to standardize the composition and effectiveness of MSC therapeutics. Numerous clinical trials have utilized mesenchymal stem cells to rejuvenate tissue and regulate the immune system. Cellular heterogeneity of MSCs slows their translation into the clinic by contributing to variable trial results. Cell-to-cell variability in the therapeutic properties of MSCs arises in vivo, differs among donors and tissues, and is compounded by inconsistencies in biomanufacturing.

Recently, CD264 was discovered to be a biomarker for aging cells in heterogeneous cultures of human bone marrow MSCs. CD264 is a decoy receptor that inhibits cell death induced by tumor necrosis factor-related apoptosis-inducing ligand. CD264⁺ MSCs exhibit an aging phenotype and diminished stem cell fitness relate to CD264⁻ MSCs. The content of CD264⁺ cells is variable in early-passage MSC cultures. CD264 is a marker of an early stage of MSC aging, which is upregulated concurrently with p21 and remains elevated as aging progresses to senescence. Culture-matched CD264⁻ and CD264⁺ MSCs have similar in vivo survival.

Therefore, there is a need for molecular profiles of MSC heterogeneity to regulate cell composition and manufacture MSC therapies with predictable treatment outcomes.

### SUMMARY OF THE DISCLOSURE

The gene signature described here can be exploited to identify molecular targets to isolate MSCs having higher proliferation and differentiation potential, and therefore more suitable for MSC therapy. Cellular aging occurs in vivo from homeostasis, trauma, and disease. It also occurs ex vivo as a cell culture is passaged, as during the biomanufacturing of MSC therapies. Cellular aging impairs the regenerative potential of MSCs to repair and restore tissue function. The ability to identify and remove early aging MSCs that are otherwise undetectable using current technologies, especially in early-passage MSCs, and the ability to identify and isolate the non-aging MSCs would produce MSCs therapeutics with more consistent and effective treatment outcomes.

Additionally, recent improvements in the nonviral delivery of CRISPR/Cas9 components have enabled safe and effective gene editing of hard-to-transfect MSCs. By genetically editing MSCs to express reporter genes corresponding to selected gene markers, it allows more efficient identification and isolation among a heterogeneous pool of MSCs through cell sorting techniques.

Once the gene signature (one or more markers having differential expression level) is identified, it enables the selection of non-aging MSCs during early passages, and the expansion thereof ensures higher homogeneity of MSCs having high proliferative and differential potentials.

In one aspect of this disclosure, a method of preparing a therapeutic mesenchymal stem cells composition is described, the method comprises: a) sorting a heterogeneous pool of MSCs based on an expression profile, and b) selecting the MSCs according to a gene signature, wherein the gene signature comprises at least one of MAP1A⁻ and PTTG1⁺.

In another aspect of this disclosure, a method of identifying early aging MSCs in cultured MSCs is described, comprising the steps of: selecting cells having a gene signature of MAP1A⁺, PTTG1⁻.

In another aspect of this disclosure, a therapeutic composition is described, the composition comprises: a therapeutically effective amount of mesenchymal stem cells (MSCs), wherein the MSCs are selected for having a gene signature and cultured and expanded ex vivo; and a therapeutically acceptable carrier; wherein the gene signature comprises at least one of MAP1A⁻, and PTTG1⁺ comparing to that of CD264⁺ MSCs.

In one embodiment, the gene signature comprises at least one of CD264⁻, MAP1A⁻, and PTTG1⁺.

In one embodiment, the gene signature comprises at least one of CD264⁺, MAP1A⁺, and PTTG1⁻.

In one embodiment, the method of preparing a therapeutic MSCs composition further comprises the step of: isolating MSCs having at least one of the following: CD264⁺, MAP1A⁺, and PTTG1⁻.

In one embodiment, the gene signature further comprises ANKRD1⁻, CDKN1A⁻, and CDKN2A⁻.

In one embodiment, the gene signature further comprises CDCA7⁺, CDK1⁺, CDKN2C⁺, and E2F1⁺ comparing to that of CD264⁺ MSCs.

In one embodiment, the gene signature is determined using Lease Absolute Shrink and Selection Operator (LASSO).

In one embodiment, the MSCs are genetically modified to include a reporter gene corresponding to genes in the gene signature.

In one embodiment, the reporter gene encodes a fluorescent protein. In one embodiment, the reporter gene encodes green fluorescent proteins (GFP), red fluorescent proteins (RFP), blue fluorescent proteins (BFP), yellow fluorescent proteins. In one embodiment, the reporter gene encodes one of the following: GFP, mCherry, mScarlet, mScarlet-I, phycoerythrin (PE), and beta-galactosidase.

In one embodiment, the MSCs in the therapeutic composition having CD264⁻, MAP1A⁻, PTTG1⁺ comprises more than 80% of all cells in the composition.

In one embodiment, the MSCs in the therapeutic composition has a colony forming unit of 35% or greater.

As used herein, except for referring to CD264+/−, the superscript “+” refers to upregulation of the gene expression, whereas the superscript “−” refers to downregulation of the gene expression. The measurement of upregulation or down regulation is not limited, as long as the quantification is acceptable to persons skilled in the art. When referring to CD264 expression profile in MSCs, the + or − refers to the population having predominantly cells having positive or negative expression of CD264 (further explained hereinafter).

In one embodiment, the up-/down-regulation of genes are measured using log fold change, which quantifies the extent of change in a gene's expression between two groups, as shown in Table 1.

In another embodiment, the up-/down-regulation of genes are measured and quantified using adjusted p-value (p_{adj}), which is a p-value corrected for multiple testing, as shown in Table 1.

In one embodiment, the up-/down-regulation of gene expression is measured by differential expression analysis using Z-scores, as shown in FIG. 4. Z-score represents how many standard deviations a gene's expression level in a sample is away from the mean expression level across all samples for that gene. The Z-score is calculated by subtracting the mean expression level of a gene across all samples from the expression level of that gene in a specific sample, and then dividing the result by the standard deviation of the gene's expression across all sample. A positive Z-score indicates higher expression than the average, a negative Z-score indicates lower expression, and a Z-score of zero means the expression is at the average.

In one embodiment, the gene expression is measured and quantified using normalized counts, which are raw read counts adjusted to account for variations in sequencing depth and gene length, in order to accurately compare gene expression levels between samples and within a single sample.

The term “gene signature” refers to a combination of expression profile of genes, which can be upregulated or downregulated or a combination of both.

The term “mesenchymal stem cells” refers to multipotent stem cells that can differentiate into various cell types, including bone cells (osteoblasts), cartilage cells (chondrocytes), muscle cells (myocytes), and fat cells (adipocytes). The primary function of MSCs is to respond to injury and infection by secreting and recruiting a range of biological factors, as well as modulating inflammatory processes to facilitate tissue repair and regeneration.

The term “early passage” of MSCs refers to passages 1-5 of cultured MSCs, and more particularly passages 2-5 of cultured MSCs.

The term “colony forming unit” refers to a measure of viable cells in a sample, typically with the unit CFU/mL.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations are used herein:

| ABBREVIATION | TERM |
|---|---|
| ANKRD1 | ankyrin repeat domain-containing protein 1 |
| CCMA | Culture medium with antibiotics |
| CDCA7 | cell division cycle-associated protein 7 |
| CDK1 | cyclin-dependent kinase 1 |
| CDKN1A | cyclin-dependent kinase inhibitor 1A |
| CDKN2A | cyclin-dependent kinase inhibitor 2A |
| CDKN2C | cyclin-dependent kinase inhibitor 2C |
| DAVID | Database for annotation, visualization and |
| | integrated discovery |
| DEG | Differentially expressed genes |
| E2F1 | E2F transcription factor 1 |
| GAGE | Generally applicable gene-set enrichment |
| GFP | Green fluorescent protein |
| GO | Gene Ontology |
| KEGG | Kyoto Encyclopedia of Genes and Genomes |
| LASSO | Least Absolute Shrinkage and Selection Operator |
| MAP1A | microtubule associated protein 1A |
| MSC | Mesenchymal stem cells |
| PE | phycoerythrin |
| PTTG1 | PTTG1 regulator of sister chromatid separation, |
| | securin |
| UniProt | Universal Protein Resource |

### BRIEF DESCRIPTION OF THE DRAWINGS

FIG. 1. Quality assessment of FACS-sorted MSCs for RNA sequencing. A-C Representative histograms (A, B) and scatter plot (C) of passage P4 MSCs (18-20 cumulative doublings) labeled with anti-CD264⁻ PE monoclonal antibody (n=10,000 cells). A Isotype (black) and parent culture (light gray). B-D CD264⁻ and CD264⁺ MSCs were immediately re-analyzed after sorting for CD264 expression (B) and scatter properties (C, D). D Scatter ratio of sorted MSCs relative to parent culture. Data are averaged over two technical replicates per CD264 group for each donor (SEM≤7% of the mean, n=5 donors). Mean values depicted as bars. E, F Representative images of sorted MSCs stained with crystal violet. G-I Efficiency of sorted MSCs to form colonies was evaluated in 10 cm cell culture dishes. I Colony-forming efficiency of CD264^{-/+} MSCs was determined for five donors. Data presented as the mean±SEM of n=5-6 dishes per donor, with each dish counted independently by two people. Scale bar=200 μm. **p<0.001 vs. CD264⁻ MSCs. Nomenclature: FACS, fluorescence-associated cell sorting; MSC, mesenchymal stem cell; PE, phycoerythrin

FIG. 2. Differential expression analysis of CD264^{-/+} MSCs. Following paired-end mRNA sequencing of sorted CD264^{-/+} populations at passage 4, reads were mapped to the human genome and analyzed for differential expression between donor-matched pairs from n=5 donors. Down- and upregulated genes, pathways and GO terms for CD264⁺ MSCs are presented relative to CD264⁻ control. A Volcano plot highlighting genes with |log 2 (fold change)|>1 and BH p_{adj}<0.1. B Table illustrating the effects of constraining p_{adj} and/or log 2 (fold change) on gene number. C, D Gene raw counts were analyzed by GAGE: dot plots of (C) KEGG pathways with BH p_{adj}<0.1 and (D) the most significantly downregulated GO terms with BH p_{adj}<0.1. All terms downregulated in CD264⁺ populations are in blue and upregulated in CD264⁺ populations are in red. ªRibosome biogenesis in eukaryotes. Nomenclature: BH, Benjamini-Hochberg; Bon, Bonferroni; ECM, extracellular matrix; FC, fold change; GAGE, Generally Applicable Gene-set Enrichment; GO, Gene Ontology; KEGG, Kyoto Encyclopedia of Genes and Genomes; MSC, mesenchymal stem cell

FIG. 3. Comparison of differential gene expression in CD264^{-/+} MSCs with that from previous bulk RNAseq studies of early-vs. late-passage MSCs. Donor-matched cultures of CD264⁻ and CD264⁺ MSCs were at passage P4 (blue). Raw RNAseq data were downloaded from the National Center for Biotechnology Information Gene Expression Omnibus repository for P2-4 vs. P11-13 MSCs from Fernandez-Rebollo et al. (2020, GSE125632, green), Hänzelmann et al. (2015, GSE59966, pink) and Wang et al. (2021, GSE178514, orange). All datasets were generated from human bone marrow MSCs and were subject to the same mapping and differential expression analysis. Venn diagrams of (A) downregulated and (B) upregulated genes with BH p_{adj}<0.1 in CD264⁺ MSCs and late-passage MSCs relative to their respective controls. (C), (D) GAGE analysis of all differentially expressed genes that are fully shared in all four datasets. Dot plots of the most significantly downregulated KEGG pathways (C) and GO terms (D) for both CD264⁺ and late-passage MSCs. No KEGG pathways or GO terms were significantly upregulated for the fully shared genes. See FIG. 2 caption for nomenclature.

FIG. 4. Validation of differential expression of LASSO-selected genes in CD264^{-/+} MSCs. A Comparison of differential expression of MAP1A and PTTG1 among all four datasets in FIG. 3. DESeq2 estimates of mean log (fold change) in gene expression with standard error for CD264⁺ vs. CD264⁻ MSCs and late-vs. early-passage MSCs. B, C Differential expression of (B) MAP1A and (C) PTTG1 in FACS-sorted CD264⁻ and CD264⁺ populations assessed by RNAseq and qPCR on independent sets of donor MSCs at passage 4. Donor-matched RNAseq raw counts and qPCR cycle threshold numbers were normalized to the same scale with centered Z-scores. Mean values depicted as bars. D, E Separation of MSCs into distinct CD264⁺ and CD264⁻ groups in quadrants Q1 and Q3, respectively, based on the combined Z-scores of MAP1A and PTTG1 for (D) RNAseq and (E) qPCR datasets. SEM<5% of the mean for RNAseq dataset and depicted as bars centered on the mean for qPCR dataset. PUM1: housekeeping gene for qPCR. Sample size: n=5 donors/dataset. **p≤0.01 vs. CD264⁻ MSCs. Nomenclature: FACS, fluorescence-activated cell sorting; LASSO, Least Absolute Shrinkage and Selection Operator; MAP1A, microtubule-associated protein 1A; MSC, mesenchymal stem cell; PTTG1, pituitary tumor-transforming gene 1; Q, quadrant; qPCR, quantitative reverse transcription polymerase chain reaction; RNAseq, RNA sequencing

FIG. 5. Applications of molecular profiles of MSC heterogeneity. Surface markers, gene signatures and other global profiles identify cell subsets with specific regenerative properties in heterogeneous MSC cultures. Molecular profiles of MSC heterogeneity have application as molecular targets and quality attributes in the production of MSC therapeutics. Targeted molecules can be regulated by small-molecule and biologics pharmaceuticals, as well as by precision gene editing. Quality attributes enable enrichment of a MSC population and its assessment during all stages of biomanufacturing of MSC therapies from cell isolation to culture expansion to storage. This control over the composition and function of MSC therapies has the potential to improve treatment outcomes for patients.

FIG. 6. Project overview to develop a gene signature of aging MSC at early passage.

FIG. 7. Predicting CD264 classification of MSCs with gene expression of MAP1A, ANKRD1, CDKN1A, CDKN2A and TP53. Positive and negative predictive values for each gene of interest were evaluated with a confusion matrix constructed from DESeq2 normalized counts. The confusion matrix compared the actual CD264 classification of MSC samples with the predicted classification based on gene expression (n=10 RNA sequencing samples). Positive predictive value=true positives/(true positives+false positives). Negative predictive value=true negatives/(true negatives+false negatives).

FIG. 8. Predicting CD264 classification of MSCs with gene expression of PTTG1, CDCA7, CDK1, CDKN2C, E2F1 and RB1. Positive and negative predictive values for each gene of interest were evaluated with a confusion matrix constructed from DESeq2 normalized counts as described in the caption for FIG. 7 (n=10 RNA sequencing samples).

### DETAILED DESCRIPTION

RNA sequencing (RNAseq) is employed to identify genes to be used in concert with CD264 as a predictive profile of aging MSCs at an early passage. Gene expression profiles reveal differences in cell populations that are undetectable with an immunophenotype alone. A gene profile can be a better predictor of cell function than immunophenotype, and two distinct cell types can have nearly identical immunophenotype. It is therefore beneficial to understand the underlying changes in pathway expression in CD264−/+ MSCs, as the genes and pathways identified here have utility as potential quality metrics to standardize biomanufacturing of MSC therapies and molecular targets to slow/reverse cellular aging.

Previous RNAseq profiling of aging MSCs investigated differential gene expression between early- and late-passage cultures, but one problem with this approach is that late passage is not clinically relevant. Typically, only early-passage MSCs are employed for therapeutic applications: two to five passages are the norm. Another problem is that the MSC cultures were heterogenous. Variation in culture composition could have obscured changes in gene expression and/or viability for therapeutic applications.

The use of CD264 as an aging marker enables for the first time the study of differential gene expression between defined populations of aging and robust cells in the same culture of early-passage MSCs. Furthermore, a composition of MSCs that provides good therapeutic efficacy and quality can be prepared based on the instant disclosure.

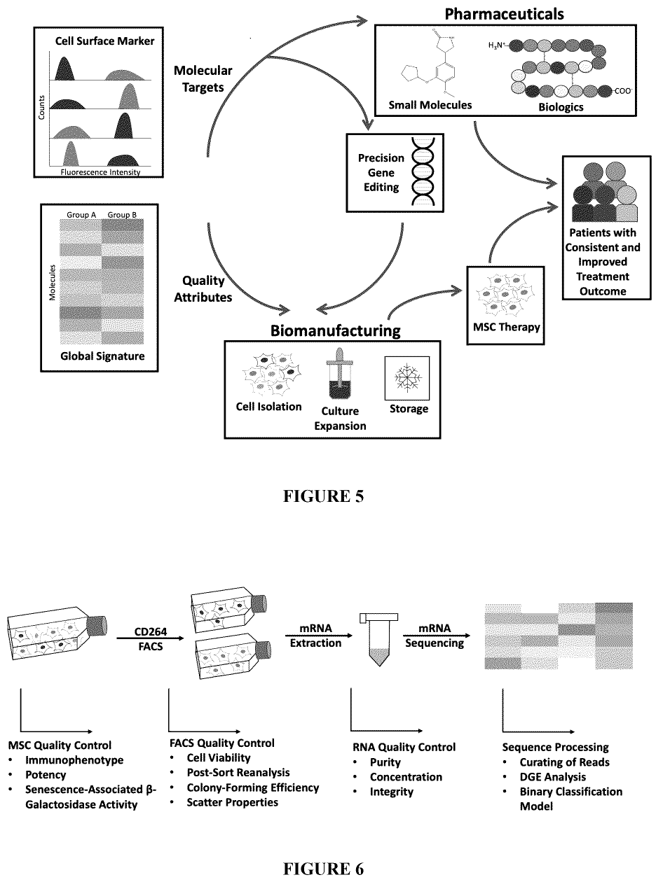

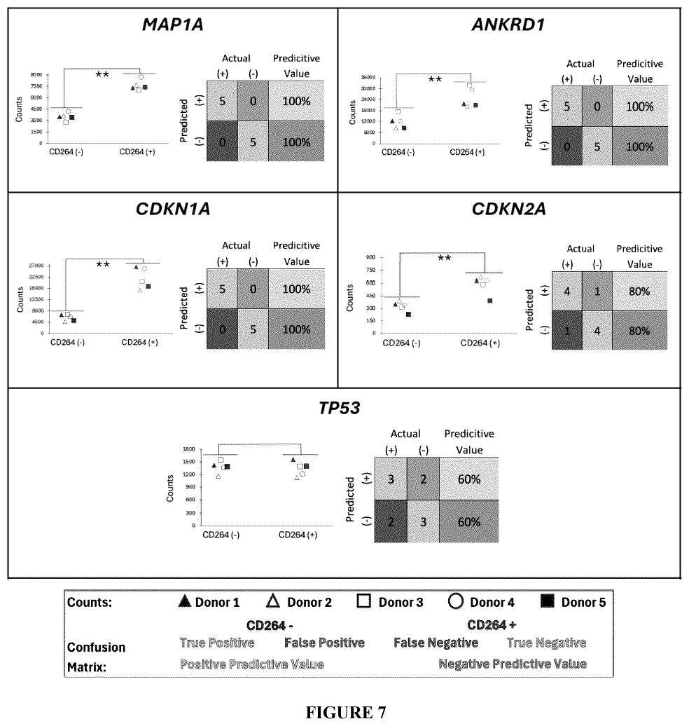

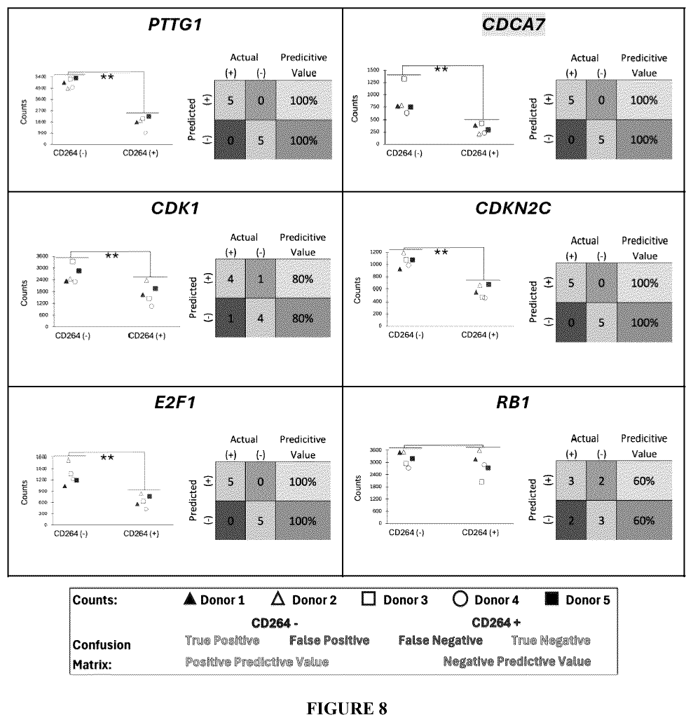

Predictive molecular profiles of the regenerative potential of MSCs have utility as quantifiable attributes of cell quality during the manufacturing of MSC therapies (FIG. 5). These quality attributes can enable enrichment of a MSC population and its assessment during all stages of manufacturing from the selection of the source stem cell to the preparation of the final clinical grade product. When measured in real time, these quality attributes can enable feedforward and feedback control of the manufacturing process to improve the consistency and quality of an MSC therapy. Most likely, MSC quality attributes will be multivariate and specific to the tissue of origin, with a unique combination of surface markers and global signatures for each application. Specifically, the global scope of transcriptomic profiling can detect differences in cell populations not evident with immunophenotyping with just cell surface markers. Different cell types can have the same immunophenotype. In these cases, a global molecular signature provides greater control over cell composition than can be achieved with an immunophenotype alone.

The present disclosure was made with the process described in FIG. 6. Heterogeneous MSCs at passage 4 were subjected to fluorescence-activated cell sorting (FACS) to isolate culture-matched CD264⁺ and CD264⁻ populations from five donors. The extracted mRNA was subjected to next-generation sequencing and subsequent computation analysis of differential gene expression (DGE) and selection of the gene signature. Differential expression of the signature genes was experimentally verified on an independent set of MSC cultures from five additional donors.

Once the expression profile (gene signature) of early aging MSCs has been identified, this disclosure further proposes the application of the gene signature. The ability to identify, sort, select early aging MSCs among a heterogeneous pool of cells, as well as expanding the selected MSCs ex vivo/in vitro enables a therapeutic composition comprising a high percentage of high quality (non-aging) MSCs. Early passage MSCs are most commonly used for therapeutic purposes due to their robust proliferation and trilineage potential. This disclosure shows that even in early passage MSCs there are still cells showing signs of aging (towards senescence), and the gene signature identified in this disclosure allows for an efficient way of sorting and selecting MSCs that are not aging yet and then expanding them into a therapeutically useful cell population. The sorting, isolation and expansion of the MSCs allow them to The genes identified in this disclosure not only provide highly accurate predictive value towards aging, but also a roadmap allowing practitioners to prepare therapeutically useful compositions. Specifically, the selected and expanded MSCs will be shown as having high proliferation and trilineage potency comparing to a heterogeneous MSC pool. This allows

### Characterization of CD264−/+ MSCs

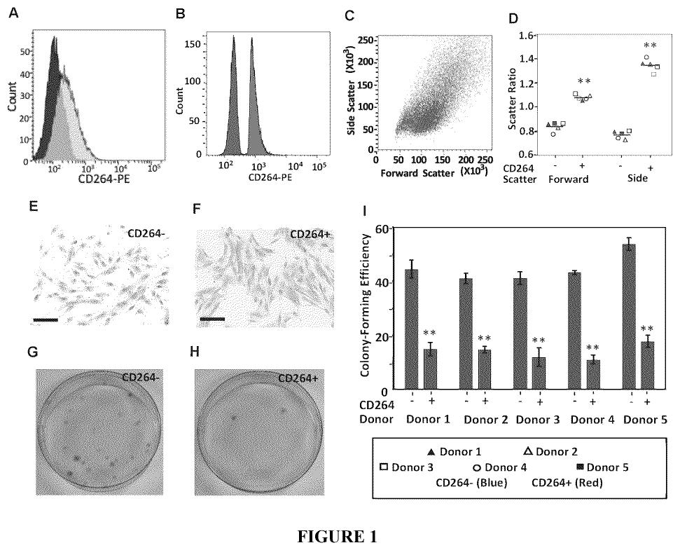

Human bone marrow MSCs from 10 donors were divided into a sequencing and validation set. The cells exhibited the immunophenotype, potency and plastic adherence that typify human MSCs. P4 MSCs (18-20 cumulative doubling) from 24- to 37-year-old donors were investigated, because passage 4 is representative of MSCs in clinical trials, and because this age group produces MSCs containing a mixture of CD264− and CD264+ cells. CD264+ cell content in the heterogeneous MSC cultures was 40% on average, consistent with previous findings. This heterogeneity enabled pairwise comparisons of differential gene expression in donor-matched CD264− and CD264+ cell populations generated by FACS. Sorted CD264− and CD264+ MSC populations are defined here as having a CD264+ cell content of <1% and >95%, respectively, by flow cytometric analysis (FIGS. 1A and B). FACS-sorted CD264+ MSCs exhibited an aging phenotype with an enlarged, granular morphology (FIG. 1C-F), less colony formation (FIG. 1E-G) and greater senescence-associated β-galactosidase (SA β-gal) activity than their CD264⁻ counterpart.

### Identification of Differential Gene and Pathway Expression in CD264−/+ MSCs

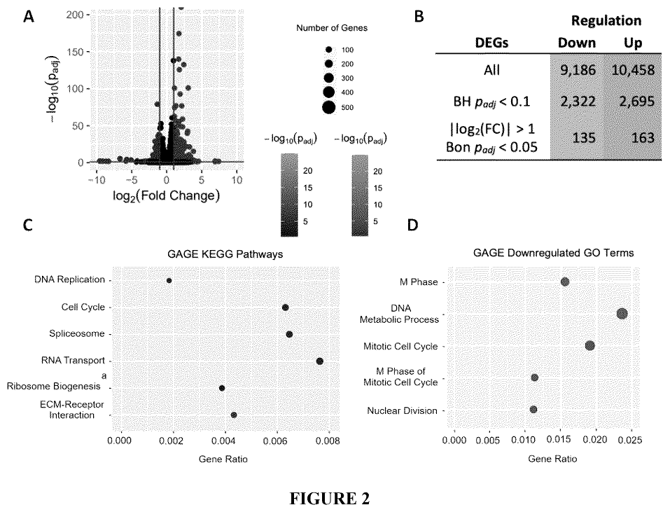

Sequencing mRNA from sorted P4 MSCs produced paired-end reads that mapped to the human genome with a 94-96% efficiency. We followed a well-established RNAseq workflow for differential gene expression and downstream analysis of CD264−/+ MSCs. The count matrix generated from the reads was analyzed with DESeq2 to detect DEGs in donor-matched CD264− and CD264+ MSCs. DESeq2 is recognized for its high sensitivity and precision in predicting DEGs. DESeq2 determined that 2,322 genes were downregulated and 2,695 genes were upregulated in CD264⁺ MSCs relative to their CD264⁻ counterpart (PH p_{adj}<0.1, FIGS. 2A and B). Of those genes, 135 downregulated genes and 163 upregulated genes in CD264⁺ MSCs satisfied the stringent threshold of a |log 2 (fold change)|>1 and Bonferroni p_{adj}<0.05 (FIG. 2B).

GAGE analysis identified how individual genes cooperated to differentially regulate pathways. This method was chosen for its ability to detect statistically and biologically relevant regulated pathways. GAGE identified six differentially regulated KEGG pathways in sorted P4 CD264^{-/+} MSCs (BH p_{adj}<0.1, FIG. 2C). DNA replication and cell cycle were among the downregulated pathways in CD264⁺ MSCs, in agreement with the slower proliferation previously reported for this cell population. The remaining downregulated pathways in CD264⁺ MSCs were associated with RNA processing at the level of ribosome biogenesis, splicing and transport. Downregulated RNA processing in CD264⁺ MSCs is consistent with the causal role of ribosome biogenesis in cell proliferation, dysregulation of splicing factor expression in senescent cells, and impaired nuclear export of mRNA during cellular aging. Extracellular matrix-receptor interaction was the only significantly upregulated KEGG pathway in CD264⁺ MSCs relative to donor-matched CD264⁻ MSCs. The composition of the extracellular matrix influences cell entrance into senescence, and conversely senescent cells cause changes to the matrix.

Functional annotation analysis supports the differentially expressed pathways in FIG. 2C. GAGE (FIG. 2D) and DAVID (data not shown) identified DNA replication and the cell cycle among the most significantly enriched terms in downregulated DEGs in CD264⁺ MSCs relative to CD264⁻ MSCs. DAVID identified several highly significant terms related to downregulated RNA processing in P4 CD264⁺ MSCs, such as mRNA splicing and the spliceosome (data now shown). Only DAVID analysis detected terms with BH p_{adj}<0.1 for upregulated DEGs in CD264⁺ MSCs. The most significant of which include cell adhesion, collagen binding and the extracellular matrix (data not shown), which support the upregulated GAGE pathway for extracellular matrix-receptor interactions in CD264⁺ MSCs (FIG. 2C).

Comparison of RNAseq Data from CD264^{-/+} MSCs and Early/Late-Passage MSCs

DEGs in P4 CD264⁺ MSCs vs. donor-matched CD264⁻ MSCs at the same passage were compared to DEGs in late-passage cultures of heterogeneous MSCs vs. early-passage MSCs reported in previous bulk RNAseq studies. Raw RNAseq data was downloaded from the Gene Expression Omnibus repository for P2-4 vs. P11-13 MSCs from Fernandez-Rebollo et al. (2020, GSE125632), Hänzelmann et al. (2015, GSE59966) and Wang et al. (2021, GSE178514). The datasets were subject to the same processing and analysis used for CD264^{-/+} MSCs to avoid artifacts from different algorithms.

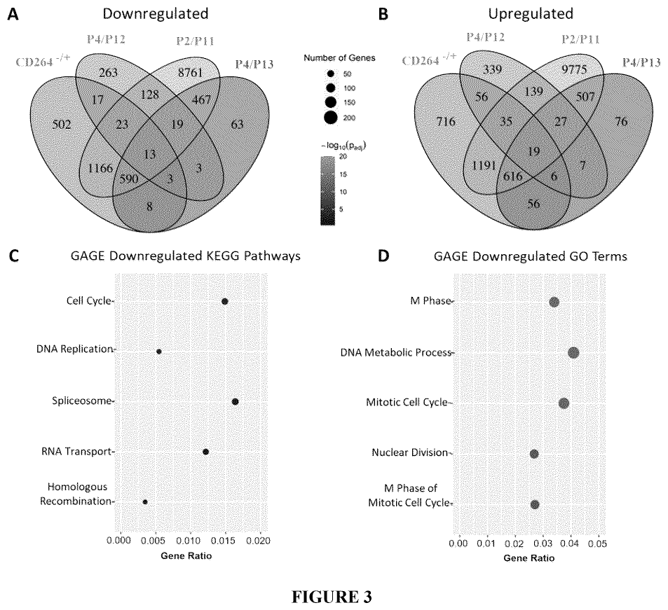

Thirteen downregulated and 19 upregulated DEGs in P4 CD264⁺ MSCs were fully shared with late-passage MSCs in the three previous datasets, and 502 downregulated and 716 upregulated DEGs were unique to CD264^{-/+} MSCs (BH p_{adj}<0.1, FIGS. 3A and B, Online Resource 3). Most of the downregulated pathways and GO terms in FIG. 3 for the fully shared DEGs are the same as in FIG. 2 for all DEGs in CD264^{-/+} MSCs. These findings indicate that impaired DNA replication, cell cycle and RNA processing are common features of aging CD264⁺ MSCs at passage 4 and senescing MSCs at passage 11-13. GAGE analysis did not detect any significantly upregulated pathways or GO terms for the fully shared DEGs, and DAVID analysis did not detect any significantly enriched terms for these DEGs.

For DEGs unique to P4 CD264−/+ MSCs, DAVID analysis indicated that ubiquitination was prominent among the most significant biological process GO terms for downregulated DEGs in CD264⁺ MSCs relative to CD264⁻ controls. Dysfunction of the ubiquitin-proteasome system is a major driver of cellular aging and may have contributed to the accumulation of aging CD264⁺ cells in our P4 MSC cultures. Extracellular matrix was the subject of several of the most significant DAVID terms for upregulated DEGs in CD264⁺ MSCs. Aging may have altered interactions of CD264⁺ MSCs with their microenvironment through the extracellular matrix. GAGE analysis was unable to identify any significantly regulated pathways or GO terms for DEGs unique to CD264^{-/+} MSCs.

### Selection and Validation of Predictive Genes for CD264^{-/+} MSCs

Predictive genes were previously selected for other cell systems by applying an arbitrary cut-off to a statistical ranking. This approach does not account for multicollinearity among genes whose expression are highly correlated. To overcome this problem, we selected predictive genes with LASSO regression, which controls multicollinearity by shrinking coefficients for features with minor effects to zero. LASSO has accurately identified predictive genes and other features for a variety of cell systems.

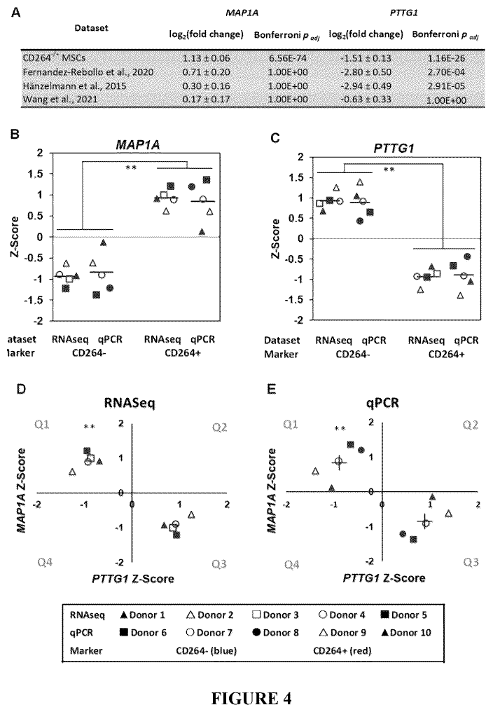

LASSO regression was performed on genes with a |log 2 (fold change)|>1 and a Bonferroni p_{adj}<0.05 as estimated by DESeq2. This log 2 (fold change) was chosen because it is a standard threshold of significance for gene expression that is verifiable via quantitative reverse transcription polymerase chain reaction (qPCR). The Bonferroni p_{adj} was used because it is more stringent than the BH p_{adj}, and thus, limits analysis to genes with the most significant change in expression. Two datasets were used for LASSO input: all DEGs in P4 CD264^{-/+} MSCs (298 genes, FIG. 2B) and those from the significant KEGG pathways (32 genes, FIG. 2C). For each set, 100 iterations of LASSO were run to classify RNAseq samples as originating from CD264⁻ or CD264⁺ MSCs. The selected genes minimized the misclassification error in all 100 iterations. LASSO regression selected microtubule-associated protein 1A (MAP1A) from all DEGs and pituitary tumor transforming gene 1 (PTTG1) from significant pathway DEGs. DESeq2 estimated a log 2 (fold change) of 1.1±0.1 and Bonferroni p_{adj} of 6.6 E−74 for MAP1A in P4 CD264⁺ MSCs relative to their CD264⁻ counterpart (FIG. 4A). The corresponding values for PTTG1 were −1.5±0.1 and 1.2 E−26, respectively. The same two genes were selected when a BH p_{adj}<0.1 was used instead of a Bonferroni p_{adj}<0.05 as an input threshold.

Our comparative analysis showed that PTTG1 is among the shared DEGs with |log ₂ (fold change)|>1 and a Bonferroni p_{adj}<0.05 for two of the three previous studies of early-vs. late-passage MSCs (FIG. 4A). PTTG1 was consistently downregulated in CD264⁺ and late-passage MSCs. MAP1A expression was higher for late-passage MSCs in all three previous studies, but the change in expression was insignificant (FIG. 4A). In our study alone, differential MAP1A expression had an average |log 2 (fold change)|>1 and Bonferroni p_{adj}<0.05.

Predictive capacity of the LASSO-selected genes was validated with qPCR on sorted P4 CD264−/+ MSCs from an independent set of 5 donors. RNAseq raw counts and qPCR threshold cycle numbers were centered for pairwise comparisons between sorted groups and normalized to the same scale with Z-scores to facilitate comparison between RNAseq and qPCR datasets. Relative expression of each target gene was normalized against Pumilio RNA-binding family member 1 (PUM1), a reference gene for aging studies.

RNAseq and qPCR datasets had similar Z-scores for both MAP1A and PTTG1 expression. For example, the MAP1A |Z-scores| for CD264⁺ vs. CD264⁻ MSCs were 0.93±0.04 for RNAseq and 0.84±0.22 for qPCR (FIGS. 4B and C). CD264 expression had a significant effect on Z-scores for both LASSO-selected genes (p<0.0001). There were no sex-linked differences in MAP1A or PTTG1 expression. The combination of MAP1A and PTTG1 Z-scores clustered MSC samples into distinct groups that had the correct CD264 classification with an accuracy of 100% (FIGS. 4D and E). CD264⁺ clusters were significantly separated from CD264⁻ clusters due to a difference in the location of cluster centroids (p≤0.01) and not a difference in cluster dispersion.

### Comparison of Differential Expression and Predictive Capacity of MAP1A, PTTG1 and Senescence Markers

For this comparison, marker genes were selected that had been previously investigated in senescent MSCs and that were associated with differentially expressed pathways in CD264^{-/+} MSCs (FIG. 2C). We identified 10 existing senescence markers that satisfied these criteria (Table 1), and they are all involved in the downregulated cell cycle pathway in CD264⁺ MSCs (FIG. 2C). Three of the marker genes (ANKRD1, CDKN1A and CDKN2A) in Table 1 were upregulated at passage 4 in aging CD264⁺ MSCs relative to CD264⁻ MSCs; four were downregulated (CDCA7, CDK1, CDKN2C and E2F1); and three had insignificant differential expression or were not detected by RNA sequencing in this study (CCND2, RB1 and TP53).

Table 1 shows fold change in expression and its significance, which were estimated with DESeq2 (n=10 RNA sequencing samples from 5 donors). Predictive values in Table 1 were evaluated with confusion matrices constructed from the DESeq2 normalized counts of gene expression (FIGS. S5 and S6). The confusion matrix mapped the predicted CD264 classification based on a ranking of the normalized counts to the actual CD264 classification of the MSC samples determined by flow cytometry. The positive predictive value is the percentage of positive predictions that are actual positives; the negative predictive value, the percentage of negative predictions that are actual negatives.

MAP1A and PTTG1 had comparable or better differential expression and predictive capacity than many of the existing senescence markers in CD264^{-/+} MSCs at passage 4. For the upregulated genes in Table 1, only CDKN1A had a larger log 2 (fold change) in expression (1.81±0.07) than MAP1A (1.13±0.06) and a smaller Bonferroni adjusted p-value (1.66 E−125 for CDKN1A vs. 6.56 E−74 for MAP1A). Both CNKN1A and MAP1A had positive and negative predictive values of 100% (n=10 samples). These findings are supported by our earlier work which demonstrated a strong correlation between the expression of CD264 and p21, which is encoded by CDKN1A. Relative to the downregulated senescence markers in Table 1, PTTG1 had comparable or higher log 2 (fold change) in expression (−1.51±0.13), the smallest Bonferroni p_{adj} (1.16 E−26), and perfect predictive values for the 10 samples examined. Notably, six of the ten senescence markers in Table 1 failed to satisfy the thresholds of differential expression (|log 2 (fold change)|>1) and/or significance (Bonferroni p_{adj}<0.05) that we used in our LASSO analysis to select MAP1A and PTTG1 as predictive genes of early passage CD264−/+ MSCs.

With RNAseq and LASSO analysis, we identified MAP1A and PTTG1 as predictive genes to distinguish between CD264⁻ and aging CD264⁺ cells in P4 MSC cultures. Neither gene was discussed in published papers from the previous RNAseq studies of early-vs. late-passage MSCs. Interrogation of the data from these studies revealed similar trends in expression for both genes during passaging as reported here. Only in our study, however, did changes in MAP1A expression exceed accepted norms for thresholds in fold change and significance. MSC heterogeneity in the previous studies may have concealed differential MAP1A expression. Our detection of MAP1A as a predictive gene of CD264⁺ MSCs demonstrates the utility of our experimental de-sign to discover novel aging genes by using well-defined cell populations.

### Predictive Genes of MSCs at an Early Stage of Aging

MSC aging is a continuous and organized process that culminates in senescence (Wagner et al., 2008). The present disclosure identified MAP1A and PTTG1 as predictive genes of a population of aging MSCs that have not become fully senescent. We previously showed that CD264 is a marker of an early stage of MSC aging: CD264⁺ MSCs undergo cell division, albeit slowly, and CD264 is upregulated before p16 during serial passage of MSCs. Possibly, aging MSCs may be more effectively rejuvenated before they senesce than afterwards since there is less cellular dysfunction. If so, MAP1A and PTTG1 would be useful to identify an MSC population amenable to rejuvenation and as potential targets to restore lost cellular function. Currently, there are several focuses on MSC rejuvenation such as mitochondrial function, oxidative stress, and chronic inflammation. Rejuvenation of MSCs can be accomplished in several ways, including genetic modification, microRNA treatment, preconditioning modification, extracellular vesicles (EVs), metabolites, or even mechanical conditioning.

Comparison with Existing Senescence Markers

Differential expression and predictive ability of MAP1A and PTTG1 compared favorably with that of existing senescence markers expressed in early passage CD264^{-/+} MSCs. The fold change in expression, its significance and predictive value of PTTG1 equaled or surpassed those of all senescence markers examined which were downregulated in CD264⁺ MSCs relative to CD264⁻ MSCs. CDKN1A was the only upregulated senescence marker whose differential expression exceeded that of MAP1A in both fold change and significance. More than half of the senescence markers investigated had expression that was below the thresholds in this study. These findings raise concern about employing established senescence markers to characterize cells at an early passage, such as CD264⁺ MSCs, that are aging but not yet fully senescent. Their expression, or lack thereof, may have limited usefulness. Instead, a new set of gene markers is warranted for cells at an early stage of aging.

### MAP1A

This instant disclosure is the first direct link of MAP1A to CD264, cellular aging and senescence for any cell type. MAP1A is frequently investigated in the context of neurodegenerative diseases, including Alzheimer's and Parkinson's. The MAP1A protein is localized in the cytosol of the cell and is mainly expressed in the brain. MAP1A is involved in microtubule assembly. We looked for any publications relating MAP1A to CD264, stem cells, aging and/or senescence. The only mentions of MAP1A with aging compare the expression of MAP1A in diseased mice at middle or late stages of aging (Jiao et al., 2020; Ma et al., 2014). There were no comparisons of non-diseased animals at different stages of aging. Additionally, we were unable to find any publications directly linking MAP1A expression to CD264, stem cells or senescence.

Insight into the differential expression of MAP1A in CD264^{-/+} MSCs comes from its function to promote microtubule polymerization in the cytoskeleton. Microtubule stabilization induces senescence in multiple cell types. Dynamic changes to the microtubule network are essential to chromosome movement during cell division. Also, microtubules influence the cellular stress response by sequestering stress response proteins and transmitting stress signals through cytoskeletal remodeling. Perhaps MAP1A upregulation in CD264⁺ MSCs stabilizes microtubules to impede cell division and/or induces a stress response and cell survival during cellular aging. The latter is consistent with the potential role of CD264 in the survival of aging MSCs.

### PTTG1

The literature is silent on the relationship of PTTG1 to CD264. PTTG1 is often examined for its role in cancer. PTTG1 is a member of the KEGG cell cycle pathway. It promotes accurate chromosome segregation during cell division by preventing premature separation of sister chromatids. There is conflicting data in the literature about PTTG1 expression in MSCs and fibroblasts. We observed that PTTG1 is downregulated in aging CD264⁺ MSCs. In support of our results, MSCs from PTTG1-null mice exhibited several key indicators of aging. Similar trends were observed by Menicanin et al. (2010) and Lee et al. (2014). Contrary to our findings, Hsu et al. (2010) found that PTTG1 overexpression induced a senescent phenotype in normal human fibroblasts. A possible explanation for this apparent contradiction could be that deviation in PTTG1 expression from the norm by either down- or upregulation may lead to genetic instability and cellular aging from erroneous chromosome segregation.

The instant disclosure provides insight as to accurately identify early passage MSCs that are senescent or approaching senescence, in order to either remove the senescent MSCs or rejuvenate them. A combination of gene and surface marker expression can form a more robust molecular profile of a cell population than an immunophenotype alone. MAP1A and PTTG1 expression will be used in concert with CD264 surface expression to identify aging cells in early-passage MSC cultures. This molecular profile has potential as a quality attribute to assess and control cell composition at all stages of MSC manufacturing from cell source selection, development of culture conditions, removal of aging cells during culture expansion, and evaluation of the final clinical product. Another application of our work is to identify molecular targets to slow and/or reverse cellular aging in MSCs. Differentially expressed genes and pathways in CD264^{-/+} MSCs can be targeted with pharmaceuticals or precision gene editing. For example, a small-molecule activator of the cell cycle pathway could be a candidate rejuvenation drug for aging CD264⁺ MSCs to counteract the downregulation of PTTG1 and other cell cycle genes. The net effect of quality assessment and molecular targeting is the potential for unprecedented control over the composition and effectiveness of MSCs therapies to improve patient outcomes.

### ANKRD1

ANKRD1 (ankyrin repeat domain-containing protein 1, or Cardiac ankyrin repeat protein) is a protein that in humans highly expressed in cardiac and skeletal muscle, and is a transcription factor involved in development and under conditions of stress. ANKRD1 has been reported to promote bone marrow-derived MSCs osteogenic differentiation by activating the Wnt signaling pathway through modulating CAV3 expression. While there has been report on elevated ANKRD1 expression in early senescence in bone marrow, there is no report on linking ANKRD1 with CD264⁺ in early passage MSCs. In this disclosure, ANKRD1 has a Log 2 (fold change) of 1.11, giving it a predictive value of 100 on both positive and negative prediction.

### CDKN1A

CDKN1A, also known as p21 or CDK-interacting protein 1, is a cyclin-dependent kinase inhibitor (CKI) that plays a crucial role in regulating the cell cycle and responding to DNA damage, primarily by inducing cell cycle arrest, direct inhibition of DNA replication and regulating apoptosis. The present disclosure establishes that in CD264⁺ MSCs CDKN1A expression is also elevated, making it a candidate for predicting cell aging/senescence.

### CDKN2A

CDKN2A, also known as cyclin-dependent kinase inhibitor 2A, is a tumor suppressor gene that regulates cell growth and division. It has two key proteins, p16^{INK4A} and p14^{ARF}, both of which act as tumor suppressors. p16 inhibits cyclin dependent kinases 4 and 6, and thereby activates the retinoblastoma family of proteins, which block traversal from G1 to S-phase. p14^{ARF} activates the p53 tumor suppressor. Activation of the CDKN2A locus promotes the cellular senescence tumor suppressor mechanism. As senescent cells accumulate with aging, expression of CDKN2A increases exponentially with aging in all mammalian species tested to date, and has been argued to serve as a biomarker of physiological age. However, a recent survey of cellular senescence induced by multiple treatments to several cell lines does not identify CDKN2A as belonging to a “core signature” of senescence markers. There was no report on the correlation between CDKN2A and CD264 in MSC aging. Here from Table 1, it is seen that in CD264⁺ early passage MSCs, CDKN2A has a Log ₂ (fold change) of 0.84, although not as high as MAP1A, still provides predictive value of 80 for both positive and negative prediction.

### CDCA7

The literature is silent on the relationship of CDCA7 to CD264. CDCA7 encodes the cell division cycle-associated protein 7, which is a transcription factor that regulates cell proliferation. Here CDCA7 is downregulated in CD264⁺ MSCs as having a Log ₂ (fold change) of −1.47 and predictive values of 100 for both positive and negative prediction, making it a good predictor for early aging MSCs.

### CDK1

CDK1, or cyclin-dependent kinase 1, is a crucial protein kinase that acts as a central regulator, driving cells through the G2 phase and into mitosis. When bound to its cyclin partners, CDK1 phosphorylation leads to cell cycle progression. While it has been reported to be a senescence marker for MSCs, CDK1 has not been correlated with CD264 in MSC early aging, particularly in early passages. CDK1 is downregulated in CD264⁺ MSCs, having a Log ₂ (fold change) of −0.69 and predictive value of 80 for both positive and negative prediction.

### CDKN2C

CDKN2C, also known as p18INK4C, is a cyclin-dependent kinase inhibitor 2C. It regulates cell cycle progression, specifically by inhibiting CDK4 and CDK6 activity to of cyclin-dependent protein serine/threonine kinase activity. Its activation is part of the transition of G1/S cell cycle. Here CDKN2C is downregulated in early passage CD264⁺ MSCs with a Log ₂ (fold change) of −0.91, and predictive values of 100 for both positive and negative predictions.

### E2F1

E2F1, also known as E2F transcription factor 1, is a transcription factor that plays a role in the control of cell cycle and action of tumor suppressor proteins. The relationship between E2F1 and CD264 expression in early passage MSCs has not been reported. Here E2F1 is down regulated in early passage CD264⁺ MSCs with Log ₂ (fold change) of −1.04. This has 100 predictive value for both positive and negative prediction.

TABLE 1
Comparison of differential gene expression and predictive
values for MAP1A, PTTG1 and senescence markers in
CD264^{-/+} MSCs at passage 4

| | Log₂ (fold | | Predictive values^{b} (%) | |
|---|---|---|---|---|
| Gene | change)^{a} | Bonterroni p_{adj}^{a} | Positive^{c} | Negative^{d} |

Upregulated^{e}

| | | | | |
|---|---|---|---|---|
| MAP1A | 1.13 ± 0.06 | 6.56E−74 | 100 | 100 |
| ANKRD1 | 1.11 ± 0.08 | 1.99E−36 | 100 | 100 |
| CDKN1A | 1.81 ± 0.07 | 1.66E−125 | 100 | 100 |
| CDKN2A | 0.84 ± 0.11 | 7.36E−11 | 80 | 80 |

Downregulated^{e}

| | | | | |
|---|---|---|---|---|
| PTTG1 | −1.51 ± 0.13 | 1.16E−26 | 100 | 100 |
| CDCA7 | −1.47 ± 0.14 | 1.81E−21 | 100 | 100 |
| CDK1^{f} | −0.69 ± 0.14 | 2.08E−02 | 80 | 80 |
| CDKN2C | −0.91 ± 0.11 | 1.97E−12 | 100 | 100 |
| E2F1 | −1.04 ± 0.12 | 6.19E−13 | 100 | 100 |

Insignificant fold change or not detected

| | | | | |
|---|---|---|---|---|
| CCND2 | Not detected | | | |
| RB1 | −0.16 ± 0.09 | 1.00E+00 | 60 | 60 |
| TP53 | −0.05 ± 0.08 | 1.00E+00 | 60 | 60 |

^{a}Fold change and adjusted p-value estimated with the DESeq2 package v1.34.0.
^{b}Predictive values for n = 10 samples from confusion matrices in FIGS. S5 and S6.
^{c}Positive predictive value = true positives/(true positives + false positives)
^{d}Negative predictive value = true negatives/(true negatives + false negatives)
^{e}Expression in CD264⁺ MSCs relative to CD264⁻ MSCs.
^{f}Also known as CDC2.

### Example 1: MSC Modification

MSCs are genetically modified to include a reporter gene for MAP1A expression, which releases fluorescent signal when MAP1A is expressed. The insertion of the MAP1A reporter gene is well known in the field and will not be repeated herein.

MSCs are further genetically modified to include a reporter gene for PTTG1 expression, which releases fluorescent signal when PTTG1 is expressed. The insertion of the PTTG1 reporter gene is also well known in the field and will not be repeated herein.

The reporter genes for MAP1A and PTTG1 are not limited, as long as they can be safely inserted and accurately report the expression of the target genes. Non-limiting example of reporting genes include GFP (green fluorescent protein), mCherry, etc. Additional reporting genes may be inserted for more genes of interest, such as ANKRD1, CDKN1A, CDKN2A, CDCA7, CDK1, CDKN2C, E2F1.

With the reporter genes emitting signals when the target genes are expressed, the MSCs can be correctly sorted using technologies such as FACS (fluorescence-activated cell sorting). This can be combined with phycoerythrin (PE) to detect surface expression of CD264 with monoclonal antibodies against CD264. The three-color reporter combination of GFP (green), mCherry (red) and PE (orange) facilitates the sorting of MAP1A, PTTG1 and CD264 expression in real time with flow cytometry.

### Example 2: MSC Quality

Cultured MSCs will be first screened for the gene expression profile of CD264, MAP1A and PTTG1 to determine the senescence state of the MSCs. MSCs with lower differential expression of PTTG1 and higher differential expression of MAP1A exhibit an aging phenotype with enlarged size, granular morphology, reduced proliferation potential and greater senescence-associated β-galactosidase activity. This cell population also has lower differentiation potential, depositing less calcified matrix during osteogenesis and producing less lipids during adipogenesis. Conversely, MSCs with higher differential expression of PTTG1 and lower differential expression of MAP1A exhibit a robust phenotype of small size, agranular, increased proliferation potential, negligible senescence-associated β-galactosidase activity and greater differentiation potential.

### Example 3: MSC Stemness and Efficacy

Cultured MSCs are selected for the differential gene expression profile of CD264, MAP1A and PTTG1. The resulting MSCs are then tested for their stemness (differentiation potential), proliferation potential (colony-forming) and optionally, efficacy. The differentiation potential of MSCs is determined 21 days after inducing osteo- and adipogenesis. Osteogenesis is induced by adding dexamethasone, β-glycerophosphate, and L-ascorbic acid 2-phosphate to culture medium. Adipogenesis is induced by adding dexamethasone, isobutylmethylxanthine and indomethacin to culture medium. Alizarin Red S (Sigma-Aldrich) identifies calcified extracellular matrix in osteogenic samples, and AdipoRed (Lonza, Walkersville, MD, USA) stains lipid droplets in adipogenic samples.

The efficiency of MSCs to form colonies containing ≥50 cells is evaluated by plating 100±10 cells in a 10-cm cell culture dish with 15 ml cell culture medium containing antibiotics. The cells are cultured undisturbed for 14 days and then stained with crystal violet (Sigma-Aldrich) in methanol (Sigma-Aldrich) to visualize colonizes.

The Senescence Cells Histochemical Staining Kit (Sigma-Aldrich) is used to assess senescence-associated β-galactosidase activity in subconfluent MSCs at pH 6 in 12-well plates. Stained cells are imaged with a BioTek Cytation 5 Cell Imaging Multimode Reader (Agilent Technologies, Santa Clara, CA, USA) in bright field mode at 4× magnification. To estimate the percentage of cells positive for senescence-associated β-galactosidase activity n≥250 cells per sample are counted in randomly selected areas of the wells. MSCs at pH 5 is the positive control for senescence-associated β-galactosidase activity.

A Gallios analyzer equipped with Gallios software v1 (Beckman Coulter, Brea, CA, USA) is used to measure senescence-associated β-galactosidase activity in living cells with flow cytometry. Unless otherwise stated, all supplies are ordered from Thermo Fisher Scientific (Waltham, MA, USA). MSCs are trypsinized and resuspended in complete culture medium with antibiotics (CCMA) at a concentration of 1-3×106 cells/ml. The cells are incubated at 37° C. in CCMA containing the pH modulator bafilomycin A1 (Sigma-Aldrich, St. Louis, MO, USA) at a concentration of 0.1 μM for 1 hour. Next, the β-gal substrate 9H-(1,3-dichloro-9,9-dimethylacridin-2-one-7-yl) β-D-galactopyranoside (DDAO galactoside) is added to the cells at a concentration of 0.02 mM, and the cells are incubated at 37° C. for an additional hour. MSC samples are then washed twice with PBS and labeled with PE-conjugated CD264 monoclonal antibodies. Labeled cells are washed twice with PBS and resuspended in PBS at a concentration of 1-2×106 cells/ml for analysis. MSC samples are processed in tandem with unlabeled and isotype controls that were treated with bafilomycin A1. Spectral overlap is corrected by multicolor compensation.

The MSCs in a therapeutic composition may comprise at least 80% of MSCs having at least one of CD264⁻, MAP1A⁻, and PTTG1⁺. The MSCs of this gene signature may have a colony-forming efficiency of 35% or better, making them ideal for MSC therapy.

The isolated and expanded (ex vivo/in vitro) MSCs according to the gene signature exhibit superior, quantifiable proliferation and differentiation potential compared to the pre-isolation and pre-expansion MSCs isolated from donors. The isolated and expanded MSCs are therefore different from the heterogeneous MSCs prior to isolation and expansion.

### Example 4: CD264+ MSCS

Prolonged in vivo survival of aging CD264⁺ MAP1A⁺ PTTG1⁻ MSCs can be used to exploit the potential therapeutic effects of the senescence-associated secretory phenotype (SASP). Senescence dramatically changes the bioactive molecules secreted by cells to produce a characteristic SASP: a complex mixture of proinflammatory cytokines, chemokines, growth factors and proteases. A growing body of data describe potential therapeutic applications of the SASP, such as the treatment of liver fibrosis, wound healing, immune cell recruitment and tissue regeneration. Creating an implantable construct containing aging CD264⁺ MAP1A+PTTG1⁻ MSCs could be a reliable method to consistently deliver the SASP to a targeted tissue.

### Materials and Methods

### MSC Cultures

Primary MSCs were harvested from human bone marrow aspirate of healthy donors after approval by the institutional review boards at Tulane University, Texas A & M University and Baylor Scott & White Hospital. Passage 0 (P0) designates plastic adherent MSCs that have not been expanded. Unless otherwise stated, all supplies were ordered from Thermo Fisher Scientific (Waltham, MA, USA). MSCs were inoculated into T-flasks at a density of 100 cells/cm² and maintained in a humidified incubator at 37° C. with 5% CO₂. The cultures were expanded in complete culture medium with antibiotics (CCMA), which consisted of minimum essential medium alpha supplemented with 20% fetal bovine serum, 2 mM L-glutamine, 100 units/ml penicillin and 100 μg/ml streptomycin. Medium was completely exchanged every 3-4 days. MSCs were harvested from culture for passaging or analysis before they reached 50% confluency. Exposure to 0.25% trypsin/1 mM EDTA for 3 min was used for cell dissociation from the culture surface. Cell viability was evaluated with trypan blue exclusion and was ≥90%. MSCs were expanded to passage 4 for flow cytometric analysis and fluorescence-associated cell sorting (FACS) into CD264^{-/+} populations as described in Online Resource 1.

### RNA Preparation and Sequencing

RNA extraction was performed immediately after sorting. P4 MSCs were washed with PBS and centrifuged at 2000 g for 12 minutes. The supernatant was discarded, and RNA was extracted with the RNeasy Plus Mini Kit (Qiagen, Germantown, MD, USA). The freshly extracted RNA was treated with RNase-Free DNase Set (Qiagen). Two technical replicates from each sorted culture were combined and then purified with RNAClean XP beads on the SPRIStand magnetic tube stand (Beckman Coulter, Brea, CA, USA). Total RNA was assessed for purity by quantifying the 260 nm/230 nm and 260 nm/280 nm ratios using the DS-11 Spectrophotometer/Fluorometer (DeNovix, Wilmington, DE, USA). RNA integrity number was measured with the TapeStation 4150 (Agilent Technologies, Santa Clara, CA, USA) using an Agilent RNA ScreenTape.

Library preparation and sequencing were done by the Tulane NextGen Sequencing Core. The TruSeq Stranded mRNA Sample Preparation Kit (Illumina, San Diego, CA, USA) was used to purify poly-A mRNA from 0.3 μg total RNA per sample and then to convert fragmented mRNA into cDNA libraries containing TruSeq RNA CD indexes (Illumina). Final cDNA libraries were quantitated using the Qubit dsDNA HS assay kit. The size and concentration of the libraries were determined by running each library on the Agilent TapeStation 4150 using the Agilent D1000 ScreenTape. Smear analysis was performed using Agilent TapeStation Software v4.1.1 with a range of 200-600 base pairs to determine the average size of each library. All libraries were pooled at a final concentration of 750 pM with a spike-in of 1% PhiX control library v3 (Illumina) and loaded on an Illumina NextSeq P2 300 reagent cartridge.

Paired-end and dual indexing sequencing (150×8×8×150 base pairs) was performed on the NextSeq 2000 (Illumina), yielding ˜66 million paired-end reads per sample. Fastq files generated by Illumina BaseSpace DRAGEN Analysis Software v1.2.1 were used for further data analysis.

### Count Matrix Generation

Our RNAseq data was submitted to the National Center for Biotechnology Information Gene Expression Omnibus (accession number GSE247950), and RNAseq data from early- and late-passage MSCs was downloaded for comparative analysis (GSE59966, GSE125632, GSE178514). The FastQC program (www.bioinformatics.babraham.ac.uk/projects/fastqc/) was used to check the overall quality of the RNAseq raw data (fastq files). FastQC and downstream analysis were performed with default parameters, unless indicated otherwise. The Salmon program v1.9.0 and the human reference transcriptome (Homo_sapiens.GRCh38.cdna.all.fa) were employed for transcript quantification analysis of the fastq files. A raw count matrix of gene expression was then generated with Bioconductor's tximport package.

### Differential Expression Analysis

The DESeq2 package v1.34.0 was used for pairwise analysis of the count matrix from donor-matched CD264⁻ and CD264⁺ samples. The package utilizes the Wald statistic to identify differentially expressed genes between samples. The Generally Applicable Gene-set Enrichment (GAGE) method in the Bioconductor package was employed to discover pathways that were differentially regulated in CD264⁻ and CD264⁺ samples. GAGE accesses the Kyoto Encyclopedia of Genes and Genomes (KEGG) database as a pathway reference. Using the raw count matrix as input, GAGE performs a meta-test that summarizes the t-test statistics for differential expression of each gene in a pathway. Up- or down-regulated genes were submitted to the Database for Annotation, Visualization and Integrated Discovery (DAVID) to identify enriched annotation terms from the KEGG pathway, Gene Ontology (GO) and Universal Protein Resource (UniProt) databases. The submitted genes had a Benjamini-Hochberg adjusted p-value (BH p_{adj})<0.1, as determined by DESeq2. DAVID uses a modified version of the Fisher exact probability test to identify enriched terms.

### Gene Selection

The GLMNET package was employed to select genes for binary classification of MSC samples. The package uses penalized maximum likelihood to fit generalized linear models and the Least Absolute Shrinkage and Selection Operator (LASSO) regression analysis for variable selection and regularization. The input to the program was the set of normalized and transformed differentially expressed genes with a |log ₂(fold change)|>1 and a Bonferroni p_{adj}<0.05. DESeq2 was used to normalize the counts based on both size factor and average transcript length. The normalized counts were then transformed so that CD264⁻ and CD264⁺ counts from the same donor were centered around 0. The cv.glmnet function was run with a 10-fold cross validation and repeated 100 times in R. After each run, the penalty parameter corresponding to the minimum misclassification error was selected, and the coefficient of each gene was determined. Genes that had non-zero coefficient values in more than 90% of the runs were selected for binary classification of MSC samples.

We used next-generation mRNA sequencing (RNA-Seq) to identify differentially expressed genes in pair-wise comparisons of RNA samples from CD264⁺ and CD264⁻ MSCs isolated from the same culture (FIG. 2). The MSC cultures are at passage 4 and are from 5 donors. We have employed the computer algorithm DESeq2 to analyze differential gene expression based on a negative binomial distribution. Computational analysis with Generally Applicable Gene-set Enrichment (GAGE) and the Database for Annotation, Visualization and Integrated Discovery (DAVID) provided insight into the cell pathways and functions associated with the differentially expressed genes in CD264+/− MSCs. We employed the Least Absolute Shrink and Selection Operator (LASSO) to select the differentially expressed genes that are the most correlated to CD264 expression in our MSC samples. The LASSO-selected genes form our gene signature of CD264⁺ MSCs at early passage. We are validating the differential expression of the genes in our signature with qPCR on culture-matched, passage 4 CD264⁺ and CD264⁻ MSCs from 5 different donors.

DESeq2 determined that 5017 genes were differentially expressed between CD264+/− populations with a p_{adj}<0.1. Of those genes, 412 were upregulated in CD264⁺ cells with a log 2 (fold change)>1 and 241 were downregulated with a log 2 (fold change)<(−1). Pathway analysis was performed to identify how individual genes cooperated to differentially regulate pathways. An observed decrease in the DNA replication and cell cycle KEGG pathways was expected due to the aging phenotype of CD264+ MSCs (FIG. 3b). We also found a downregulation in pathways related to RNA processing and transport. The extracellular matrix-receptor interaction pathway was the only significantly upregulated KEGG pathway. The enriched gene ontology (GO) terms determined with DAVID (FIGS. 3c and d) are consistent with the differentially expressed KEGG pathways.

We employed LASSO to select the differentially expressed genes that are the most correlated to CD264 expression in our MSC samples. LASSO regression analysis uses penalized maximum likelihood to fit generalized linear models. This computational method preserves regression coefficients with major effects and shrinks all other coefficients into zeros according to a penalty parameter, 2. LASSO analysis was run 100 times to perform cross validation. After each run, the 2 value corresponding to the minimum misclassification error was selected, and the coefficient of each gene was determined. Genes that had non-zero coefficient values in more than 90% of the runs were selected as candidates for inclusion in the gene signature. FIG. 4 presents a representative LASSO misclassification error plot. In this example, one gene in the gene signature is sufficient to have a misclassification error of zero when predicting that a culture is composed of either CD264⁺ or CD264⁻ cells.

The following references are incorporated by reference in their entirety for all purposes.
Bertolo A, Baur M, Guerrero J, Pötzel T, Stoyanov J (2019) Autofluorescence is a reliable in vitro marker of cellular senescence in human mesenchymal stromal cells. Sci Rep 9(1):2074. doi: 10.1038/s41598-019-38546-2
Bertolo A, Guerrero J, Stoyanov J (2020) Autofluorescence-based sorting removes senescent cells from mesenchymal stromal cell cultures. Sci Rep 10(1):19084. doi: 10.1038/s41598-020-76202-2
Cai B, Shao N, Ye T, Zhou P, Si W, Song H et al (2023) Phosphorylation of MAP1A regulates hyperphos-phorylation of Tau in Alzheimer's disease model. Neuropathol Appl Neurobiol 49 (5):e12934. doi: 10.1111/nan.12934
Cao Y J, Zheng Y H, Li Q, Zheng J, Ma L T, Zhao C J, Li T (2022) MSC senescence-related genes are associated with myeloma prognosis and lipid metabolism-mediated resistance to proteasome inhibitors. J Oncol 2022:4705654. doi: 10.1155/2022/4705654
Chaput C D, Shar A, Jupiter D, Hubert Z, Clough B, Krause U, Gregory C A (2018) How stem cell com-position in bone marrow aspirate relates to clinical outcomes when used for cervical spine fusion. PLoS One 13 (9):e0203714. doi: 10.1371/journal.pone.0203714
Chu S, Moujaber O, Lemay S, Stochaj U (2022) Multiple pathways promote microtubule stabilization in senescent intestinal epithelial cells. NPJ Aging 8:16. doi: 10.1038/s41514-022-00097-8
Costa L A, Eiro N, Fraile M, Gonzalez L O, Saa J, Garcia-Portabella P et al (2021) Functional heterogeneity of mesenchymal stem cells from natural niches to culture conditions: implications for further clinical uses. Cell Mol Life Sci 78 (2):447-467. doi: 10.1007/s00018-020-03600-0
Demaria, M., Ohtani, N., Youssef, S. A., Rodier, F., Toussaint, W., Mitchell, J. R., . . . Campisi, J. (2014). An essential role for senescent cells in optimal wound healing through secretion of PDGF-AA. Developmental Cell, 31(6), 722-733. doi: 10.1016/j.devcel.2014.11.012
Dennis G Jr, Sherman B T, Hosack D A, Yang J, Gao W, Lane H C, Lempicki R A (2003) DAVID: database for annotation, visualization, and integrated discovery. Genome Biol 4 (5):3. doi: 10.1186/gb-2003-4-5-p3
Dominici M, Le Blanc K, Mueller I, Slaper-Cortenbach I, Marini F, Krause D et al (2006) Minimal criteria for defining multipotent mesenchymal stromal cells. The International Society for Cellular Therapy position statement. Cytotherapy 8 (4): 315-317. doi: 10.1080/14653240600855905
Fernandez-Rebollo E, Franzen J, Goetzke R, Hollmann J, Ostrowska A, Oliverio M et al (2020) Senes-cence-associated metabolomic phenotype in primary and iPSC-derived mesenchymal stromal cells. Stem Cell Reports 14 (2):201-209. doi: 10.1016/j.stemcr.2019.12.012
Friedman J H, Hastie T, Tibshirani R (2010) Regularization paths for generalized linear models via coordinate descent. J Stat Softw 33 (1):1-22. doi: 10.18637/jss.v033.i01
González-Bermudez L, Anglada T, Genesca A, Martin M, Terradas M (2019) Identification of reference genes for RT-qPCR data normalisation in aging studies. Sci Rep 9:13970. doi: 10.1038/s41598-019-50035-0
González-Gualda E, Baker A G, Fruk L, Muñoz-Espín D (2021) A guide to assessing cellular senescence in vitro and in vivo. FEBS J 288(1):56-80. doi: 10.1111/febs.15570
Gutierrez-Garcia G, Cardesa-Salzmann T, Climent F, González-Barca E, Mercadal S, Mate J L et al (2011) Gene-expression profiling and not immunophenotypic algorithms predicts prognosis in patients with diffuse large B-cell lymphoma treated with immunochemotherapy. Blood 117 (18):4836-4843. doi: 10.1182/blood-2010-12-322362
Hanzelmann S, Beier F, Gusmao E G, Koch C M, Hummel S, Charapitsa I et al (2015) Replicative senes-cence is associated with nuclear reorganization and with DNA methylation at specific transcription factor binding sites. Clin Epigenetics 7 (1):19. doi: 10.1186/s13148-015-0057-5
Hosack D A, Dennis G, Sherman B T, Lane H C, Lempicki R A (2003) Identifying biological themes within lists of genes with EASE. Genome Biol 4 (10):R70. doi: 10.1186/gb-2003-4-10-r70
Hsu Y H, Liao L J, Yu C H, Chiang C P, Jhan J R, Chang, L C et al (2010) Overexpression of the pituitary tumor transforming gene induces p53-dependent senescence through activating DNA damage response pathway in normal human fibroblasts. J Biol Chem 285 (29):22630-22638. doi: 10.1074/jbc.M109.096255
Ito M, Hara H, Takeda N, Naito A T, Nomura S, Kondo M et al (2019) Characterization of a small molecule that promotes cell cycle activation of human induced pluripotent stem cell-derived cardiomyocytes. J Mol Cell Cardiol 128:90-95. doi: 10.1016/j.yjmcc.2019.01.020
Jiao L, Zheng M, Duan J, Wu T, Li Z, Liu L et al (2020) Tau knockout exacerbates degeneration of parval-bumin-positive neurons in substantia nigra pars reticulata in Parkinson's disease-related alpha-synu-clein A53T mice. FASEB J 34 (9):12239-12254. doi: 10.1096/fj.202000017RR
Kang, T., Yevsa, T., Woller, N., Hoenicke, L., Wuestefeld, T., Dauch, D., . . . Zender, L. (2011). Senescence surveillance of pre-malignant hepatocytes limits liver cancer development. Nature, 479 (7374), 547-551. doi: 10.1038/nature10599
Kevei E, Hoppe T (2014) Ubiquitin sets the timer: impacts on aging and longevity. Nat Struct Mol Biol 21 (4):290-292. doi: 10.1038/nsmb.2806
Klein L E, Freeze B S, Smith A B 3rd, Horwitz S B (2005) The microtubule stabilizing agent discodermolide is a potent inducer of accelerated cell senescence. Cell Cycle 4 (3):501-507. doi: 10.4161/cc.4.3.1550
Krizhanovsky, V., Yon, M., Dickins, R. A., Hearn, S., Simon, J., Miething, C., . . . Lowe, S. W. (2008). Senescence of activated stellate cells limits liver fibrosis. Cell, 134(4), 657-667. doi: 10.1016/j.cell.2008.06.049
Latorre E, Pilling L C, Lee B P, Bandinelli S, Melzer D, Ferrucci L, Harries L W (2018) The VEGFA156b isoform is dysregulated in senescent endothelial cells and may be associated with prevalent and incident coronary heart disease. Clin Sci (Lond) 132 (3):313-325. doi: 10.1042/CS20171556
Lee H J, Choi J H, Jung J, Kim J K, Lee S S, Kim G J (2014) Changes in PTTG1 by human TERT gene expression modulate the self-renewal of placenta-derived mesenchymal stem cells. Cell Tissue Res 357 (1):145-157. doi: 10.1007/s00441-014-1874-0
Levi N, Papismadov N, Solomonov I, Sagi I, Krizhanovsky V (2020) The ECM path of senescence in aging: components and modifiers. FEBS J 287 (13):2636-2646. doi: 10.1111/febs.15282
Liu S, de Castro L F, Jin P, Civini S, Ren J, Reems J A et al (2017) Manufacturing differences affect human bone marrow stromal cell characteristics and function: comparison of production methods and products from multiple centers. Sci Rep 7:46731. doi: 10.1038/srep46731
Liu Y, Al-Adra D P, Lan R, Jung G, Li H, Yeh M M, Liu Y Z (2022) RNA sequencing analysis of hepato-cellular carcinoma identified oxidative phosphorylation as a major pathologic feature. Hepatol Com-mun 6 (8):2170-2181. doi: 10.1002/hep4.1945
Love M I, Huber W, Anders S (2014) Moderated estimation of fold change and dispersion for RNA-seq data with DESeq2. Genome Biol 15 (12): 550. doi: 10.1186/s13059-014-0550-8
Love M I, Anders S, Kim V, Huber W (2019) RNA-seq workflow: gene-level exploratory analysis and differential expression. Bioconductor. https://master.bioconductor.org/packages/release/workflows/vignettes/rnaseqGene/inst/doc/rnaseqGene.html
Luo W, Friedman M S, Shedden K, Hankenson K D, Woolf P J (2009) GAGE: generally applicable gene set enrichment for pathway analysis. BMC Bioinformatics 10:161. doi: 10.1186/1471-2105-10-161
Luo W (2024) RNA-seq data pathway and gene-set analysis workflows. Bioconductor.
https://bioconductor.org/packages/3.21/bioc/vignettes/gage/inst/doc/RNA-seqWorkflow.pdf
Madsen S D, Jones S H, Tucker H A, Giler M K, Muller D C, Discher C T et al (2020) Survival of aging CD264+ and CD264− populations of human bone marrow mesenchymal stem cells is independent of colony-forming efficiency. Biotechnol Bioeng 117 (1):223-237. doi: 10.1002/bit.27195
Madsen S D, Russell K C, Tucker H A, Glowacki J, Bunnell B A, O'Connor K C (2017) Decoy TRAIL receptor CD264: a cell surface marker of cellular aging for human bone marrow-derived mesenchymal stem cells. Stem Cell Res Ther 8 (1):201. doi: 10.1186/s13287-017-0649-4
Mastrolia I, Foppiani E M, Murgia A, Candini O, Samarelli A V, Grisendi G et al (2019) Challenges in clinical development of mesenchymal stromal/stem cells: concise review. Stem Cells Transl Med 8 (11):1135-1148. doi: 10.1002/sctm.19-0044
McHugh, D., & Gil, J. (2018). Senescence and aging: Causes, consequences, and therapeutic avenues. The Journal of Cell Biology, 217(1), 65-77. doi: 10.1083/jcb.201708092
Menicanin D, Bartold P M, Zannettino A C W, Gronthos S (2010) Identification of a common gene expression signature associated with immature clonal mesenchymal cell populations derived from bone mar-row and dental tissues. Stem Cells Dev 19 (10): 1501-1510. doi: 10.1089/scd.2009.0492
O'Connor K C (2019) Molecular profiles of cell-to-cell variation in the regenerative potential of mesenchymal stromal cells. Stem Cells Int 2019:5924878. doi: 10.1155/2019/5924878
Pan G, Ni J, Yu G, Wei Y F, Dixit V M (1998) TRUNDD, a new member of the TRAIL receptor family that antagonizes TRAIL signalling. FEBS Lett 424 (1-2):41-45. doi: 10.1016/s0014-5793(98)00135-5
Park H S, Lee J, Lee H S, Ahn S H, Ryu H Y (2022) Nuclear mRNA export and aging. Int J Mol Sci 23 (10):5451. doi: 10.3390/ijms23105451
Parker A L, Kavallaris M, McCarroll J A (2014) Microtubules and their role in cellular stress in cancer. Front Oncol 4:153. doi: 10.3389/fonc.2014.00153
Patro R, Duggal G, Love M I, Irizarry R A, Kingsford C (2017) Salmon provides fast and bias-aware quantification of transcript expression. Nat Methods 14 (4):417-419. doi: 10.1038/nmeth.4197
Pazzagli C, Avila J (1994) Purification of brain microtubule-associated protein MAP1A. Neurochem Res 19 (9):1195-1198. doi: 10.1007/bf00965155
Ren Q, Jin B (2017) The clinical value and biological function of PTTG1 in colorectal cancer. Biomed Pharmacother 89:108-115. doi: 10.1016/j.biopha.2017.01.115
Ritschka, B., Storer, M., Mas, A., Heinzmann, F., Ortells, M. C., Morton, J. P., . . . . Keyes, W. M. (2017). The senescence-associated secretory phenotype induces cellular plasticity and tissue regeneration. Genes & Development, 31(2), 172-183. doi: 10.1101/gad.290635.116
Rodriguez-Fuentes D E, Fernandez-Garza L E, Samia-Meza J A, Barrera-Barrera S A, Caplan A I, Barrera-Saldana H A (2021) Mesenchymal stem cells current clinical applications: a systematic review. Arch Med Res 52 (1): 93-101. doi: 10.1016/j.arcmed.2020.08.006
Rosati D, Palmieri M, Brunelli G, Morrione A, Iannelli F, Frullanti E et al (2024) Differential gene expression analysis pipelines and bioinformatic tools for the identification of specific biomarkers: a review. Comput Struct Biotechnol J 23:1154-1168. doi: 10.1016/j.csbj.2024.02.018
Rubinek T, Chesnokova V, Wolf I, Wawrowsky K, Vlotides G, Melmed S (2007) Discordant proliferation and differentiation in pituitary tumor-transforming gene-null bone marrow stem cells. Am J Physiol Cell Physiol 293 (3): C1082-1092. doi: 10.1152/ajpcell.00145.2007
Sekiya I, Larson B L, Smith J R, Pochampally R, Cui J G, Prockop D J (2002) Expansion of human adult stem cells from bone marrow stroma: conditions that maximize the yields of early progenitors and evaluate their quality. Stem Cells 20 (6): 530-541. doi: 10.1634/stemcells.20-6-530
Soneson C, Love M I, Robinson M D (2015) Differential analyses for RNA-seq: transcript-level estimates improve gene-level inferences. F1000Res 4:1521. doi: 10.12688/f1000research.7563.2
Stanfill B, Reehl S, Bramer L, Nakayasu E S, Rich S S, Metz T O et al (2019) Extending classification algorithms to case-control studies. Biomed Eng Comput Biol 10:1179597219858954. doi: 10.1177/1179597219858954
Tibshirani R (1996) Regression shrinkage and selection via the lasso. J R Statist Soc Series B (Methodol) 58 (1):267-288
Tighe S, Held M A (2010) Isolation of total RNA from transgenic mouse melanoma subsets using fluores-cence-activated cell sorting. Methods Mol Biol 632:27-44. doi: 10.1007/978-1-60761-663-4_2
Vega F, Chang C C, Medeiros L J, Udden M M, Cho-Vega J H, Lau C C et al (2005) Plasmablastic lymphomas and plasmablastic plasma cell myelomas have nearly identical immunophenotypic profiles. Mod Pathol 18 (6):806-815. doi: 10.1038/modpathol.3800355
Vicente J J, Wordeman L (2019) The quantification and regulation of microtubule dynamics in the mitotic spindle. Curr Opin Cell Biol 60:36-43. doi: 10.1016/j.ceb.2019.03.017
Volarevic S, Stewart M J, Ledermann B, Zilberman F, Terracciano L, Montini E et al (2000) Proliferation, but not growth, blocked by conditional deletion of 40S ribosomal protein S6. Science 288 (5473):2045-2047. doi: 10.1126/science.288.5473.2045
Wagner W, Horn P, Castoldi M, Diehlmann A, Bork S, Saffrich R et al (2008) Replicative senescence of mesenchymal stem cells: a continuous and organized Process. PLoS ONE 3 (5):e2213. doi: 10.1371/journal.pone.0002213
Wang S, Wang Z, Su H, Chen F, Ma M, Yu W et al (2021) Effects of long-term culture on the biological characteristics and RNA profiles of human bone-marrow-derived mesenchymal stem cells. Mol Ther Nucleic Acids 26:557-574. doi: 10.1016/j.omtn.2021.08.013
Wilson A, Webster A, Genever P (2019) Nomenclature and heterogeneity: consequences for the use of mesenchymal stem cells in regenerative medicine. Regen Med 14 (6):595-611. doi: 10.2217/rme-2018-0145
Yin Z Q, Liu J J, Xu Y C, Yu J, Ding G H, Yang F et al (2014) A 41-gene signature derived from breast cancer stem cells as a predictor of survival. J Exp Clin Cancer Res 33 (1): 49. doi: 10.1186/1756-9966-33-49
Zou H, McGarry T J, Bernal T, Kirschner M W (1999) Identification of a vertebrate sister-chromatid separation inhibitor involved in transformation and tumorigenesis. Science 285 (5426):418-422. doi: 10.1126/science.285.5426.418

1. A method of preparing a therapeutic mesenchymal stem cells (MSCs) composition, the method comprising the steps of:
a) sorting a heterogeneous pool of MSCs based on gene expression profiles; and
b) selecting the MSCs according to a gene signature;
c) wherein the gene signature comprises at least one of MAP1A⁻ and PTTG1⁺.

2. The method of claim 1, wherein the gene signature further comprises ANKRD1⁻, CDKN1A⁻, and CDKN2A⁻ as compared to CD264⁺ MSCs.

3. The method of claim 1, wherein the gene signature further comprises at least one of the following genes as compared to CD264⁺ MSCs: CDCA7⁺, CDK1⁺, CDKN2C⁺, and E2F1⁺.

4. The method of claim 1, wherein the gene signature is determined using Least Absolute Shrink and Selection Operator (LASSO).

5. The method of claim 1, further including:
d) isolating MSCs having at least one of: CD264⁺, MAP1A⁺, and PTTG1⁻.

6. A method of determining a gene signature for mesenchymal stem cells, comprising the steps of:
a) obtaining a differential expression profile of mesenchymal stem cells; and
b) analyzing the differential expression profile to determine a gene signature that is correlated with a target gene based on a threshold of statistical significance;
c) wherein in step b) Least Absolute Shrinkage and Selection Operator (LASSO) is employed in the analysis.

7. The method of claim 5, wherein the target gene is CD264.

8. The method of claim 5, wherein the threshold of statistical significance is smaller than 0.1.

9. The method of claim 5, wherein in step a) the expression profile is obtained by mRNA sequencing.

10. The method of claim 5, wherein the MSCs are genetically modified to include a reporter gene corresponding to the gene signature.

11. The method of claim 9, wherein the reporter gene encodes a fluorescent protein or other visible protein.

12. The method of claim 9, wherein the reporter gene encodes one or more proteins selected from a green fluorescent protein (GFP), a red fluorescent protein (RFP), a blue fluorescent protein (BFP), and/or a yellow fluorescent protein.

13. The method of claim 9, wherein the reporter gene encodes one of the following: GFP, mCherry, mScarlet, mScarlet-I, phycoerythrin (PE), and beta-galactosidase.

14. The method of claim 5, wherein the gene signature is at least one of CD264⁻, MAP1A⁻, and PTTG1⁺.

15. The method of claim 5, wherein the gene signature is at least one of CD264⁺, MAP1A⁺, and PTTG1⁻.

16. A therapeutic composition, comprising:
a) a therapeutically effective amount of mesenchymal stem cells (MSCs), wherein the MSCs are selected for having a gene signature and cultured and expanded ex vivo; and
b) a therapeutically acceptable carrier;
c) wherein the gene signature comprises at least one of MAP1A⁻, PTTG1⁺ comparing to that of CD264⁺ MSCs.

17. The therapeutic composition of claim 15, wherein the MSCs are early passage (passages 1-5) cultured MSCs.

18. The therapeutic composition of claim 15, wherein the gene signature further comprises at least one of ANKRD1⁻, CDKN1A⁻, or CDKN2A⁻.

19. The therapeutic composition of claim 15, wherein the gene signature further comprises at least one of CDCA7⁺, CDK1⁺, CDKN2C⁺, or E2F1⁺ comparing to that of CD264⁺ MSCs.

20. The therapeutic composition of claim 15, wherein the MSCs having CD264⁻, MAP1A⁻, PTTG1⁺ comprises more than 80% of all cells in the composition.

21. The therapeutic composition of claim 15, wherein the MSCs has a colony forming unit of 35% or greater.
